(19) United States
(12) Patent Application Publication
(10) Pub. No.: US 2024/0382387 A1
(43) Pub. Date: Nov. 21, 2024

(54) DENTAL COMPOSITION AND DENTINE CULTURING METHOD

(71) Applicant: AIR WATER INC., Osaka-shi, Osaka (JP)

(72) Inventors: Narifumi KISHIDA, Kobe-shi, Hyogo (JP); Misako NAKASHIMA, Kobe-shi, Hyogo (JP)

(21) Appl. No.: 18/564,869

(22) PCT Filed: Mar. 25, 2022
(86) PCT No.: PCT/JP2022/014543

§ 371 Date: Nov. 28, 2023

(30) Foreign Application Priority Data
May 28, 2021 (JP) 2021-090610

(51) Int. Cl. A61K 6/17; A61L 27/36

(52) U.S. Cl. CPC A61K 6/17; A61L 27/36

(57) ABSTRACT

Composition (4) contains fragmented materials (3) obtained by fragmenting a human tooth and is suitable as a scaffold for human dental pulp cells because the cross-section of a tooth contains dentine and dentinal tubules. Furthermore, because of a human tooth, it is possible to induce odontoblasts from human dental pulp cells and lead to the regeneration/repair of dentine without making the patient feel anxious about drugs. Therefore, by using a dental composition containing the fragmented material of a human tooth, dental pulp tissue can be repaired/regenerated, and it is easy to ensure the safety of substances used in dental pulp regeneration therapy.

### TECHNICAL FIELD

The present invention relates to a dental composition and a dentine culturing method that are used for dental caries treatment, pulp extirpation/infected root canal treatment, etc.

### BACKGROUND ART

The structure of a tooth is a layered structure in which enamel, dentine, and dental pulp are located from the outside. Among these, dentine is one of hard tissues that make up the tooth, and is composed of about 70% inorganic matter and about 30% organic matter. Dentine is characterized by being softer than enamel, which is mostly composed of inorganic substances. The soft dentine layered beneath the hard enamel acts as a cushion, so the tooth as a whole has a strong structure that resists cracking even when subjected to impact.

However, as dental caries progresses and the dentine erodes, the dental pulp beneath the dentine surface gradually becomes closer to the surface of the tooth into an exposed state in which the dental pulp is connected to the oral cavity through dentinal tubules. The dental pulp is nerve tissue, so if the dental pulp comes close to the surface of the tooth, it will cause subjective symptoms of dental caries, such as tooth sensitivity/pain. General treatment when dental caries progresses and symptoms such as dental pulpitis appear is to perform pulp extirpation to remove the inflamed dental pulp.

The dental pulp has a function of notifying the dental caries through the sense of pain, and the dental pulp tissues themselves have a buffering action, which can prevent fractures. The dental pulp thus has the role of protecting the tooth through the metabolic actions of blood vessels/nerves, anti-inflammatory/infection-preventing actions, etc. Hence, pulp extirpation increases the risk of tooth loss due to fracture, spread of infection to the apical periodontal tissues, or worsening of inflammation. If such a situation occur in a pulp-extirpated tooth, treatment options include dentures and implant treatment. These treatment methods, however, have a problem of causing a decline in aesthetics and occlusal properties. In this context, as a new treatment method, dental pulp regeneration therapy has been proposed in which the dental pulp tissues are restored to their original state after pulp extirpation. This treatment refers to a method of regenerating dental pulp tissues through collecting dental pulp tissues from an unnecessary tooth such as a wisdom tooth, culturing the dental pulp stem cells present therein, and transplanting the dental pulp stem cells and a drug into the pulp-extirpated tooth. According to this treatment, the entire dental pulp tissues are regenerated and dentine is added to the dentine wall of the root canal, but currently, only a small amount of dentine regeneration can be induced in the coronal portion, and it is reinforced with a covering or filling such as cement, resin, or metal.

Regarding approaches to regenerating dentine tissues, a chemical scheme has been proposed in which drugs are added to induce differentiation of dental pulp cells. For example, Patent Document 1 describes a method of regenerating dentine by differentiating dental pulp cells into odontoblasts using as a scaffold a composite material in which non-collagen phosphorylated proteins such as phosphorin are crosslinked to collagen. Patent Document 2 describes a dentine formation promoter that contains an HGM-CoA reductase inhibitor as an active ingredient. Patent Document 3 describes a method of producing differentiated and induced odontoblasts that includes culturing isolated human dental pulp cells three-dimensionally to a predetermined cell density while adding 1,25 (dihydroxy) vitamin D3. Unfortunately, however, as with these chemical approaches, odontoblasts induced to differentiate in vitro are terminally differentiated, so they cannot adhere even when transplanted in vivo, and it is considered to be difficult to use them for dentine regeneration.

In contrast to the chemical approach which is a scheme of preparing odontoblasts and then transplanting them, a method adopting a physical approach has been proposed, using an inorganic calcined artificial material to differentiate dental pulp cells into odontoblasts in a pulp-extirpated tooth. For example, Patent Document 4 describes a method of regenerating dentine using a ceramic porous carrier. However, the ceramic porous carrier described in this document has a problem in that it is extremely small in diameter, 100 to 500 μm, and is not practical from the viewpoint of handling and transplantation.

### PRIOR ART DOCUMENTS

### Patent Documents

PATENT DOCUMENT 1: WO2005/079728
PATENT DOCUMENT 2: WO2008/120720
PATENT DOCUMENT 3: JP4884678B
PATENT DOCUMENT 4: JP2005-270647A

### OBJECT AND SUMMARY OF THE INVENTION

The present invention has been made in view of such problems, and an object of the present invention is to provide a dental composition and a dentine culturing method that are suitable for dental pulp regeneration therapy in which the safety of the substances used can be readily ensured.

The dental composition according to the present invention has the following features.
<1> A dental composition used for promoting regeneration of dentine, the dental composition comprising a fragmented material of a human tooth.
<2> The dental composition according to <1>, wherein the fragmented material has a particle diameter of 500 to 2000 μm.
<3> The dental composition according to <1>, wherein the fragmented material is subjected to a demineralization process.
<4> The dental composition according to <1>, wherein the fragmented material is subjected to a demineralization process using a 0.4 to 1.0 normal strong acid aqueous solution as a demineralization liquid.
<5> The dental composition according to <1>, wherein the fragmented material is subjected to a demineralization process using an aqueous solution of an ethylenediamine derivative as a demineralization liquid.
<6> The dental composition according to <1>, wherein the fragmented material is immersed in a solution containing an antibacterial agent and then dried.
<7> The dental composition according to <6>, wherein the solution containing the antibacterial agent is an alcohol solution containing silver ions.

The dentine culturing method according to the present invention has the following features.
<8> A dentine culturing method comprising bringing dental pulp cells into contact with a dental composition to induce odontoblasts, the dental composition containing a fragmented material of a human tooth.

The present invention uses the fragmented material of a human tooth as a component that promotes regeneration of dentine. It is therefore possible to provide a dental composition that can readily ensure the safety and that can be suitably used for dentine regeneration therapy that repairs/regenerates capped dentine on the regenerated dental pulp tissue surface during the dental pulp regeneration therapy or directly on the dental pulp surface during dental pulp capping/vital dental pulp cutting treatment.

### BRIEF DESCRIPTION OF DRAWINGS

FIG. 1 is a schematic diagram illustrating the positions of fragmented materials of a tooth in the treatment of a pulp-extirpated tooth;

FIG. 2A is a CBCT image of Example 1;

FIG. 2B is a CBCT image showing an enlarged view of region A of FIG. 2A;

FIG. 3A is a tissue specimen photograph of Example 1;

FIG. 3B is a tissue specimen photograph showing an enlarged view of region B of FIG. 3A;

FIG. 4A is a schematic diagram of dental pulp regeneration therapy using a dental composition containing fragmented materials of a tooth at a stage immediately after transplantation;

FIG. 4B is a schematic diagram of dental pulp regeneration therapy using a dental composition containing fragmented materials of a tooth at an advanced stage of dentine regeneration;

FIG. 5 is a set of schematic diagrams for describing the progress after dental pulp transplantation in dental pulp regeneration therapy;

FIG. 6 is a set of explanatory diagrams illustrating the correspondence between schematic diagrams at 4 to 6 months and 12 months after transplantation and tissue specimen photographs of Example 1;

FIG. 7A is a microscope photograph of Example 2 (demineralization process, 3 wt % EDTA);

FIG. 7B is a microscope photograph of Example 2 (demineralization process, 3 wt % EDTA);

FIG. 7C is a microscope photograph showing an enlarged view of region C of FIG. 7B;

FIG. 8 is a microscope photograph of Example 2 (physiological saline);

FIG. 9A is a microscope photograph of Example 3 (demineralization process, 0.6 normal hydrochloric acid);

FIG. 9B is a microscope photograph of Example 3 (demineralization process, 17 wt % EDTA);

FIG. 9C is a microscope photograph of Example 3 (demineralization process, 0.6 normal hydrochloric acid and then 17 wt % EDTA); and

FIG. 10 is a microscope photograph of Example 3 (undemineralized).

### BEST MODE FOR CARRYING OUT THE INVENTION

### «Dental Composition»

The dental composition of the present invention (which may also be referred to as “the composition,” hereinafter) contains a fragmented material of a human tooth (natural tooth). The cross-section of a tooth contains dentine and dentinal tubules, which are suitable as scaffolds for dental pulp cells including dental pulp stem cells, so the use of the fragmented material of a tooth can promote regeneration of dentine. From the viewpoint of compatibility, it is preferred that the dental pulp stem cells and the fragmented material of a tooth used for regeneration of dentine originate from the patient himself/herself, but they may be provided by another person. By using dental pulp stem cells and a fragmented material of a tooth provided by a donor other than the patient, it is possible to promote regeneration of dentine of a tooth, for example, in patients who do not have unnecessary teeth such as wisdom teeth, in patients whose dental pulp stem cell function has decreased due to aging, or in other similar patients. Examples of persons other than the patient include the patient's relatives, but the persons may also be those other than the patient's relatives if they are compatible with the patient. It is preferred that the dental pulp stem cells and the fragmented material of a tooth provided from a person other than the patient originate from the same tooth, but they may originate from different teeth. The following description will be made for an embodiment in which the dentine of a pulp-extirpated tooth is regenerated using dental pulp stem cells extracted from a patient's unnecessary tooth and a fragmented material of the unnecessary tooth.

Dental pulp regeneration therapy begins with the extraction of a patient's unnecessary tooth. Cultivation of dental pulp stem cells is performed after extracting the dental pulp tissues within the tooth, but in this case, the tooth fragments remaining after the extraction of dental pulp tissues have conventionally been discarded. Some of the dental pulp cells, including dental pulp stem cells, survive by adhering to the scaffold, and differentiate into odontoblasts in response to microstructures on the μm order. In addition, the dentine and dentinal tubules of tooth fragments from which human dental pulp cells have been extracted are composed of the individual patient's own proteins. It is therefore considered that the tooth fragments remaining after the extraction of dental pulp tissues have good compatibility with the dental pulp stem cells and do not cause immune rejection.

The present inventors have come up with an idea of using the above properties of the fragmented material of a tooth to utilize a conventionally discarded tooth to induce differentiation of dental pulp stem cells into odontoblasts and promote dentine regeneration/repair. The present inventors have also found that it is effective to make the cracked tooth fragments an appropriate size and keep them at appropriate positions in order to achieve the above guiding and promoting functions. Moreover, since the tooth remaining after collection of human dental pulp cells have conventionally been discarded, the dental composition can be produced at low cost by using the tooth, from which human dental pulp cells have been collected, as a component to promote regeneration of dentine.

The tooth from which human dental pulp cells have been collected has good compatibility with the dental pulp stem cells extracted as dental pulp tissues within the tooth. In addition, the proteins in dentine and dentinal tubules are unique to each individual patient. Therefore, by using a tooth from which human dental pulp cells used for dentine regeneration have been collected, it is possible to avoid immune rejection reactions, efficiently induce differentiation of human dental pulp cells into odontoblasts, and regenerate dentine.

The present invention uses a conventionally discarded tooth (unnecessary tooth), which becomes unnecessary after human dental pulp cells are collected, to induce and promote dentine regeneration. Examples of unnecessary teeth include teeth extracted during orthodontic treatment and wisdom teeth. The composition of the present invention uses a conventionally discarded tooth, and it therefore has little impact on the environment and can be produced using a very inexpensive and eco-friendly production method. Moreover, the broken pieces of a tooth, that is, fragmented materials of the tooth, can be produced at the same time as the dental pulp tissues using a press machine that splits the tooth and extracts the dental pulp tissues, and the production efficiency is good.

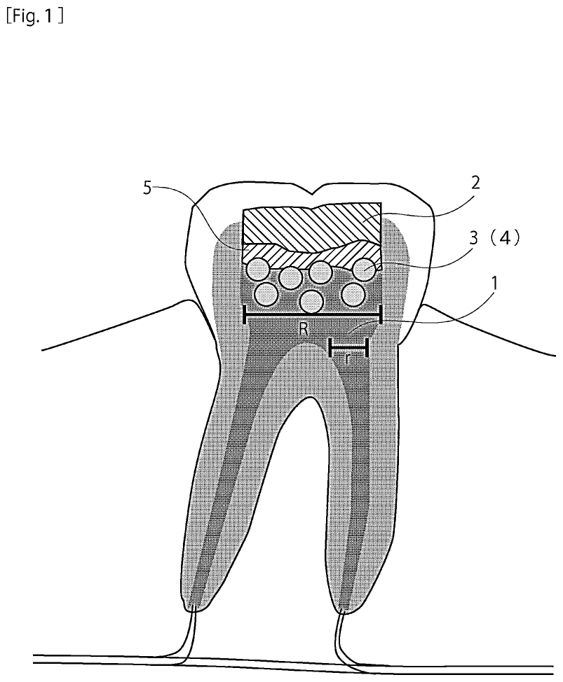

FIG. 1 is a schematic diagram illustrating the positions of fragmented materials 3 in a composition 4 in the treatment of a pulp-extirpated tooth. As illustrated in the figure, in the treatment of the pulp-extirpated tooth, a mixture 1 of dental pulp stem cells and a drug is transplanted into the pulp-extirpated tooth, and the fragmented materials 3 of a tooth contained in the composition 4 are statically placed on the mixture 1. After being covered with a dentine substitute 5, a portion from which dentine and enamel have been removed during the coronal portion treatment is reinforced with a covering material of resin 2.

Odontoblasts prepared in vitro are terminally differentiated cells, so even if they are transplanted, they do not adhere to the side wall of the root canal. The dental pulp stem cells in the mixture 1 are not terminally differentiated cells and function as a dentine ground substance. The dental pulp stem cells can therefore adhere to the side wall of the root canal when transplanted in vivo.

As the dentine substitute 5, for example, a bioceramic material such as Biodentine (registered trademark, available from Septodont), MTA (mineral trioxide aggregate) cement, or the like is used.

From the viewpoint of suppressing the movement of the fragmented materials 3 to the lower side of the mixture 1, that is, to the side far from the resin 2, the composition 4 is configured such that the particle diameter of the fragmented materials 3 placed statically on the mixture 1 is a predetermined size. This allows the positions of the fragmented materials 3 to be maintained at the coronal portion above the mixture 1. By maintaining the positions of the fragmented materials 3 in the pulp-extirpated tooth near the boundary between the mixture 1 and the resin 2, the effect of promoting regeneration of dentine can be enhanced.

From the viewpoint of inducing differentiation into odontoblasts and regenerating dentine by keeping the fragmented materials 3 in appropriate positions, the particle diameter of the fragmented materials 3 is preferably larger than the root canal diameter. The root canal diameter, however, varies depending on the location of the tooth and the age of the patient. With age, the proportion the dentine occupies increases, and the dental pulp cavity filled with dental pulp tissues and the root canal diameter narrow, resulting in a smaller root canal diameter. Therefore, the particle diameter of the fragmented materials 3 used for inducing dentine regeneration needs to be determined depending on the site of the tooth and age of the patient.

As described above, the size of a root canal diameter r varies depending on the site of the tooth and the age of the patient, but is usually about 0.5 to 1.0 mm (500 to 1000 μm). Therefore, from the viewpoint of preventing the fragmented materials 3 from falling into the root canal, the particle diameter of the fragmented materials 3 to be used may be 500 μm or more, preferably 600 μm or more, and more preferably 700 μm or more.

The particle diameter of the fragmented materials 3 may be smaller than a width (cross-sectional distance) R of the portion into which the mixture 1 of dental pulp stem cells and drug is injected, for example, 2000 μm or less. Reducing the particle diameter increases the surface area of the fragmented materials 3, which can be said to be advantageous for regeneration of dentine. In this regard, the particle diameter of the fragmented materials 3 is preferably 1500 μm or less and more preferably 1000 μm or less.

As described above, in usual cases, by setting the particle diameter of the fragmented materials 3 to 500 to 2000 μm and preferably 500 to 1000 μm, the fragmented materials 3 can be maintained at positions effective for promoting regeneration of dentine. However, since the size of the root canal diameter varies depending on the individual and site, the appropriate particle diameter is determined by the target root canal for transplantation. From the viewpoint of promoting regeneration of dentine, the particle diameter of the fragmented materials 3 is preferably equal to or larger than the root canal diameter r of the root canal as the transplantation target. For example, about 5 to 15 pieces of the fragmented materials 3 may be used for one tooth. In the present invention, a numerical range A to B means A or more and B or less.

The fragmented materials 3 for use having a particle diameter within the above range can be taken out from the fragmented tooth, for example, using a strainer (sieve). The average particle diameter (median diameter, D50) of the fragmented materials 3 contained in the composition 4 is preferably 600 to 1500 μm and more preferably 700 to 900 μm.

While the fragmented materials 3 contain an enamel portion and a dentine portion, it is mainly the dentine portion that has the effect of promoting dental pulp regeneration. Therefore, from the viewpoint of promoting regeneration of dentine, the fragmented materials 3 are preferably subjected to a demineralization process. By eluting calcium salt crystals from the enamel portion of the fragmented material 3 through the demineralization process, the effect of regenerating dentine by the dentine portion is improved. In the present invention, the demineralization process refers to dissolving calcium phosphate, which is the main component of tooth enamel.

A demineralization liquid used in the demineralization process for the fragmented materials 3 may be an acidic to weakly alkaline aqueous solution in which a ground substance is dissolved.

Demineralization with acid is a relatively strong process that uses an action of eluting calcium phosphate. Examples of the ground substance used for acid demineralization include hydrochloric acid, formic acid, nitric acid, trichloroacetic acid, sodium sulfate, sodium citrate, Plank-Rychlo's solution (mixture of hydrochloric acid, formic acid, and aluminum chloride), and Morse's solution (mixture of formic acid and sodium citrate). From the viewpoint of sufficiently eluting calcium phosphate, a demineralization liquid containing a strong acid such as hydrochloric acid or nitric acid as the ground substance is preferred.

Demineralization with a chelating agent is a relatively weak process that uses an action of softening calcium phosphate. Examples of base materials used for demineralization with a chelating agent include an ethylenediamine derivative such as sodium salt of ethylenediaminetetraacetic acid (EDTA). The ethylenediamine derivative is used, for example, as an aqueous solution of about 1 to 30 wt %.

From the viewpoint of sufficiently dissolving enamel of the fragmented materials of a tooth to expose dentine and promoting differentiation of dental pulp stem cells into tubular dentine, bone-like dentine, etc. in the pulp-extirpated tooth, a demineralization liquid that performs demineralization with an acid is preferred, and a strong acid such as hydrochloric acid is more preferred. The concentration of the strong acid aqueous solution is preferably 0.1 to 2.0 normal and more preferably 0.3 to 1.0 normal.

The time for processing the fragmented materials with a demineralization liquid may be sufficient if it can dissolve the enamel of the fragmented materials. For example, when subjecting the fragmented materials 3 having a particle diameter (D50) of about 700 to 900 μm to the demineralization process using an aqueous solution of 0.3 to 1.0 normal hydrochloric acid, the processing time is preferably 10 to 40 hours and more preferably 15 to 30 hours.

The fragmented materials 3 are preferably those that are immersed in a solution containing an antibacterial agent and then dried. Examples of the solution containing an antibacterial agent include an alcohol solution containing silver ions, pure water containing silver ions, and silica water.

The composition of the present embodiment can be produced, for example, as follows.
(1) Put a tooth into a cylinder having an open top, insert the body from above, and apply pressure to randomly fracture the tooth. For the tooth cracking member, select a metal that is hard and biocompatible so as not to be problematic even if it adheres to broken pieces of the tooth. Examples of such a metal include stainless steel/SUS and titanium alloys.
(2) Use a strainer (sieve) to collect the fragmented materials (broken pieces) of the tooth having a predetermined diameter (500 to 2000 μm).
(3) Transfer the fragmented material of the tooth having a predetermined diameter to a tube or the like and sterilize it by soaking it with disinfectant ethanol.
(4) Add pure water or the like and confirm sterility using a culture test. Soak it in disinfectant ethanol again.
(5) Dry the disinfectant ethanol in a sterile space.

### «Dentine Culturing Method»

The dentine culturing method of the present invention induces bringing a dental composition into contact with dental pulp cells to induce odontoblasts. The dental composition contains a fragmented material of a human tooth. Since a drug that induces odontoblasts is not used, there is no risk that patients to whom the cultured dentine is applied will feel anxious about the drug, and the safety can be readily ensured. Moreover, the fragmented material of a tooth used for inducing odontoblasts can be produced at the same time as pulp extirpation, and the dentine culture can therefore be carried out at low cost.

### EXAMPLES

### Example 1

The fragmented materials of a dog-derived tooth were transplanted into the root canals of two dogs (total of eight root canals) with some of the dental pulp remaining. In order to observe the progress after transplantation, images were captured using dental CT (cone beam CT, CBCT) four months after transplantation, and tissue specimens were prepared and photographed six months after transplantation.

### «Experimental Conditions»

Fragmented materials of tooth: A tooth extracted from a dog as the transplantation target was fragmented with a press machine, and a strainer was used to take out the fragmented materials (undemineralized) having a particle diameter of 500 to 1000 μm.

Transplantation target tooth: A tooth with viable dental pulp (dental pulp remaining) was used as the transplantation target.

Transplant location: 5 to 10 fragmented materials of the tooth were placed statically near the entrance of the root canal of the transplantation target tooth without using any scaffolding material other than the fragmented materials of the tooth.

### «Results»

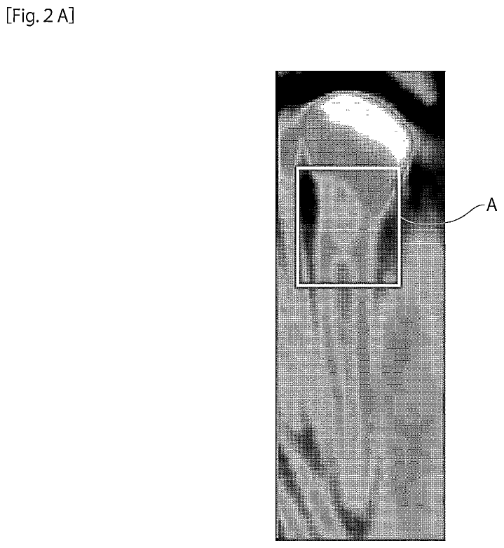

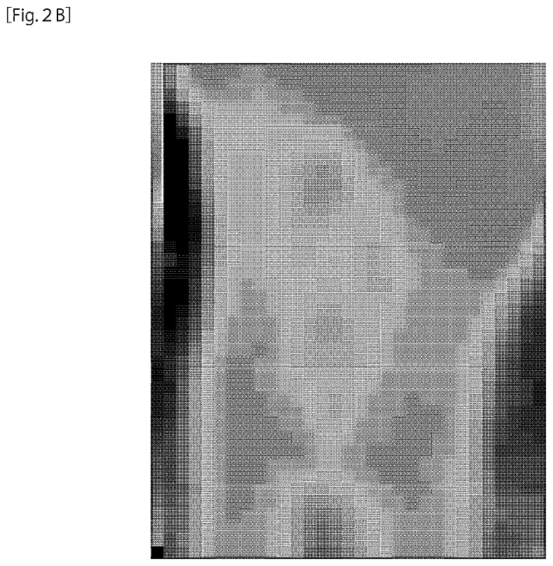

FIG. 2A is a CBCT image captured four months after transplantation, and FIG. 2B is a CBCT image showing an enlarged view of region A surrounded by a rectangle in FIG. 2A. As shown in these figures, four months after the transplantation, a white, opaque, dentine-like hard tissue was formed on the tooth to which the fragmented materials were transplanted. In addition, dentine-like hard tissues were formed in all of the teeth to which the fragmented materials were transplanted, as in the tooth shown in FIGS. 2A and 2B, four months after the transplantation.

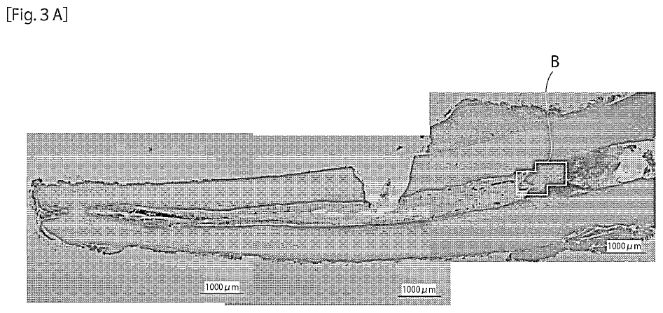

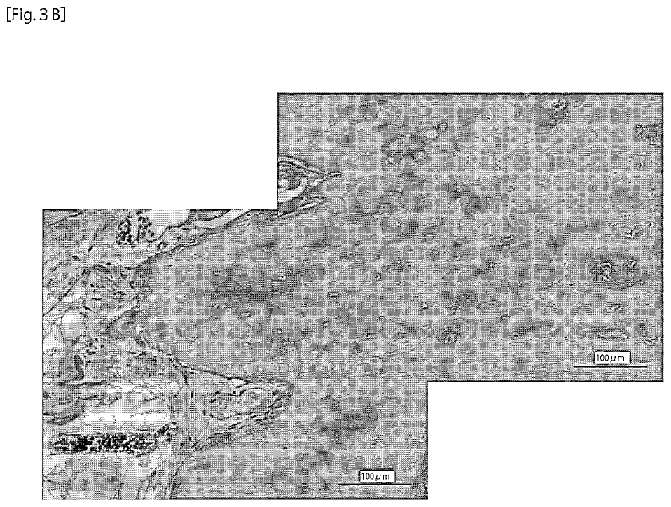

FIG. 3A is a photograph of a tissue specimen six months after transplantation, and FIG. 3B is a photograph of a tissue specimen showing an enlarged view of region B surrounded by a rectangle in FIG. 3A. As shown in FIG. 3A, six months after transplantation, a layer of bone-like dentine-like hard tissue was formed over a wide range on the regenerated dental pulp surface. Moreover, as shown in FIG. 3B, it was found that the regeneration of bone-like dentine progressed between the root canal wall surfaces. These results indicate that the fragmented materials 3 of a tooth placed near the entrance of the root canal can promote regeneration of dentine.

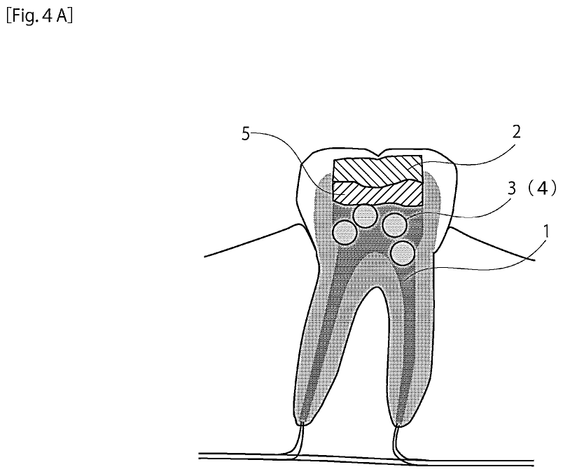

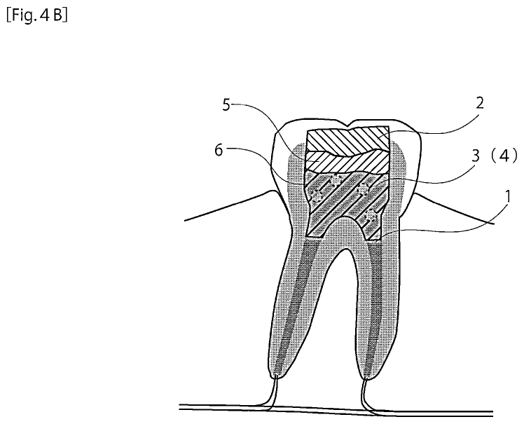

FIG. 4A is a cross-sectional view schematically illustrating the tooth at the time when the fragmented materials were transplanted, and FIG. 4B is a cross-sectional view schematically illustrating the tooth when sufficient time for regeneration has passed after the transplantation. As illustrated in FIG. 4A, by using fragmented materials having a particle diameter of 500 to 1000 μm, regenerated dentine 6 can be formed near the entrance of the root canal. As the formation of the regenerated dentine 6 progresses, the regenerated dentine 6 fills the gaps between the fragmented materials and the remaining coronal dentine, and the natural tooth and the regenerated dentine fuse together thereby to create a natural core (stuck part) beneath the resin 2. The strength of the tooth after treatment is therefore improved, and it is possible to prevent reinfection (microleakage) of bacteria from the oral cavity and re-causing the dental caries (secondary caries).

The fragmented materials 3 that promote the formation of the regenerated dentine 6 are obtained by fragmenting a tooth. The fragmented materials 3 are denatured/melted and gradually replaced with the regenerated dentine and therefore come to exhibit the same mechanical/chemical properties as those of the regenerated dentine 6 after the regenerated dentine 6 is formed. For example, the changes in thermal expansion caused by changes in environmental temperature are the same. Therefore, when eating something hot or cold, no gaps are created between the fragmented materials 3 and the regenerated dentine 6 and natural dentine. On the other hand, in the case of using artificial materials, there is a risk that gaps may form due to differences in thermal expansion when eating something hot or cold. Since autologous proteins do not cause immune rejection, it is more preferred to form the fragmented materials 3 using a tooth of the person to be treated.

FIG. 5 is a set of schematic diagrams of root canal models for describing the progress after transplantation. As illustrated in the figure, the mixture 1 of dental pulp stem cells and a drug is transplanted into a pulp-extirpated tooth, and the fragmented materials 3 are placed statically on top of the mixture 1 and then covered with the resin 2. In the figure, the dentine substitute 5 is placed between the fragmented materials 3 and the resin 2.

Four to six months after the transplantation, the regenerated dentine (regenerated bone-like dentine) 6 is formed in the region in which the fragmented materials 3 are placed statically, and regenerated dental pulp 7 is formed in the region in which the mixture 1 is transplanted. By regenerating not only dental pulp stem cells but also dental pulp tissues such as blood vessels and nerves and drawing the dental pulp tissues to the portion in which dentine is to be regenerated, it is possible to achieve dentine regeneration in which cells adhere to the root canal side surface.

Twelve months after the transplantation, tubular dentine 8 is formed beneath the regenerated dentine 6 (above the regenerated dental pulp 7) (see “Dentine induction by implants of autolyzed antigen-extracted allogenic dentine on amputated dental pulps of dogs,” Misako Nakashima, 1989).

FIG. 6 is a set of explanatory diagrams illustrating the correspondence relationships between the root canal models at 4 to 6 months and 12 months after transplantation and the tissue specimen photographs of Example 1 (6 months after transplantation). As illustrated in the figure, it can be found from the tissue specimen photograph 6 months after the transplantation that the regenerated dental pulp 7 is formed. The same photograph also shows that the formation of the tubular dentine 8 begins below the regenerated dentine 6.

### «Comparative Example 1»

The fragmented materials of a dog-derived tooth were transplanted into the root canals with some of the dental pulp remaining in the same manner as in Example 1 except that fragmented materials having a particle diameter of 150 to 500 μm were used as the fragmented materials of a tooth as substitute for the fragmented materials having a particle diameter of 500 to 1000 μm, and the progress was observed. When the fragmented materials having a particle diameter of 150 to 500 μm were used, however, no capped dentine formation was observed. This is considered to be because the fragmented materials did not stay near the entrance of the root canal and fell toward the apical side of the tooth.

It can be said to be advantageous for promoting regeneration of dentine that reducing the particle diameter increases the surface area of the fragmented materials. However, since no capped dentine formation was observed in the coronal portion, the location of the fragmented materials staying is important in order to promote regeneration of dentine, and it can be said to be necessary to keep the fragmented materials above the entrance of the root canal of the coronal portion.

In Example 1, it can be said that by using the fragmented materials 3 having a particle diameter of 500 to 1000 μm, the fragmented materials 3 remained near the entrance of the root canal, resulting in the promotion of the capped dentine formation. Moreover, the fragmented materials having a particle diameter of 500 to 1000 μm were easier to handle than those having a particle diameter of 150 to 500 μm, and were superior in the workability in transplantation.

### Examples 2 and 3

In the enamel and dentine portions that make up the fragmented materials of a tooth, the dentine portion is considered to mainly contributes to the promotion of regeneration of dentine. Therefore, the influence of processing the surfaces of the fragmented materials using a solution was evaluated by the following method. That is, dental pulp stem cells were brought into contact with fragmented materials of a tooth that were subjected to different demineralization processes, and the effect of promoting the differentiation ability, or the dentine formation ability, was evaluated.

### Example 2

### «Fragmented Materials of Tooth»

A dog-derived tooth was fragmented with a press machine, and a strainer was used to take out the fragmented materials of the tooth having a particle diameter of 500 to 1000 μm.

### «Solution and Processing Time»

The fragmented materials were used after being immersed in the following solutions for the following time, washed with water, and dried. Process (A) is a demineralization process, in which enamel is removed from the surfaces of the fragmented materials. Process (B) is a control.

(A) 3 wt % EDTA aqueous solution (Smear Clean; trade name, NIPPON SHIKA YAKUHIN CO., LTD., demineralization liquid), 30 minutes

(B) Physiological saline, 30 minutes

### «Culture»

Dental pulp stem cells (1.25×10⁶ cells) and dried tooth fragments were made into pellets (a state in which the cells and the fragmented materials overlapped at the bottom of a tube) and mixed with the following medium in a centrifuge tube, centrifugation was performed, and the pellets were cultured in the centrifuge tube without a completely sealed lid.

Medium: 2 ml of 10% fetal bovine serum (FBS)/Dulbecco's Modified Eagle's Medium (DMEM)

Medium exchange frequency: 3 to 4 days (replace ⅔ of the liquid volume to avoid damage to pellets)

Culture environment: 37° C. in CO₂ incubator

Culture period: 22 days

### «Measurement»

After culturing for about 3 weeks (22 days), the pelleted dental pulp stem cells and fragmented materials were fixed in formalin and then sectioned, and the degree of adhesion of the dental pulp stem cells to the tooth fragments was confirmed using microscope photographs.

### Example 3

### «Fragmented Materials of Tooth»

An unnecessary tooth was fragmented with a press machine, and a strainer was used to take out the fragmented materials of the tooth having a particle diameter of 750 to 1000 μm.

### «Solution and Processing Time»

The fragmented materials were used after being immersed in the following solution for the following time, washed with water, and dried. Processes (C) to (E) are demineralization processes, in which enamel is removed from the surfaces of the fragmented materials. Process (F) is a control.

(C) 0.6 normal hydrochloric acid aqueous solution, 24 hours

(D) 17 wt % EDTA aqueous solution (Smear Clean; trade name, NIPPON SHIKA YAKUHIN CO., LTD., demineralization liquid), 5 minutes

(E) Process (D) after process (C)

(F) Undemineralization

### «Culture»

Dental pulp stem cells (4.7×10⁵ cells) and dried tooth fragments were made into pellets and mixed with the following medium in a centrifuge tube, centrifugation was performed, and the pellets were cultured in the centrifuge tube without a completely sealed lid.

Medium: 2 ml of Dulbecco's Modified Eagle's Medium (DMEM) containing Porcine serum of 20 wt % based on DMEM and L-ascorbic acid (=vitamin C, differentiation promoter) of a concentration of 0.5 mg/ml

Medium exchange frequency: 3 to 4 days (replace ⅔ of the liquid volume to avoid damage to pellets)

Culture environment: 37° C. in CO₂ incubator

Culture period: 14 days

### «Evaluation of Demineralization Process»

Approximately the same number of the fragmented materials were divided into 35 mm dishes, and the difference in the degree of X-ray transmission depending on the presence or absence of demineralization was visually confirmed using a dental X-ray device KDEX-SII (KINKI ROENTGEN INDUSTRIAL CO., LTD.). As a result, it has been confirmed that the fragmented materials of (F) appear in the X-ray photograph, the fragmented materials of (C) or (E) do not appear in the X-ray photograph, and the fragmented materials of (D) slightly appear in the X-ray photograph. From these results, it has been confirmed that the enamel in the fragmented materials of (C) and (E) is sufficiently dissolved by the demineralization process and the dentine becomes the main component while a small amount of enamel remains in the fragmented materials of (D) even after the demineralization process.

### «Measurement»

After culturing for about 2 weeks (14 days), the pelleted dental pulp stem cells and fragmented materials were fixed in formalin and then sectioned, and the degree of adhesion of the dental pulp stem cells to the tooth fragments was confirmed using microscope photographs.

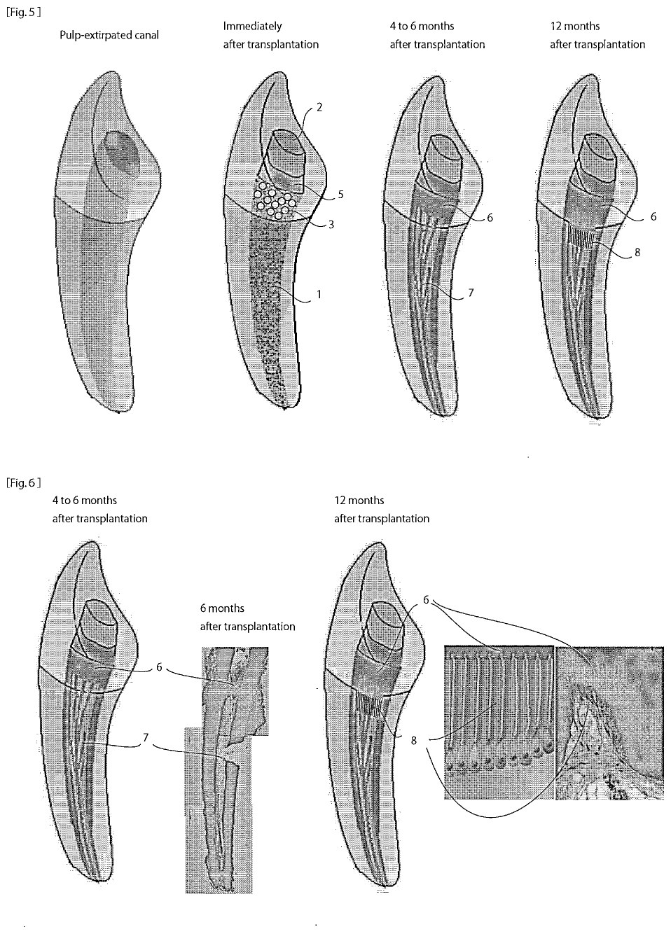

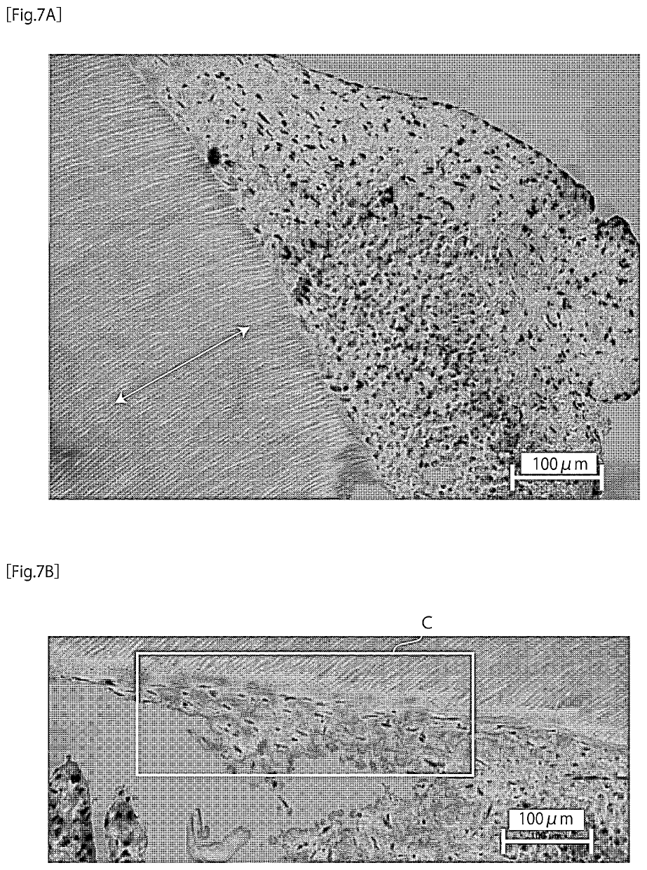

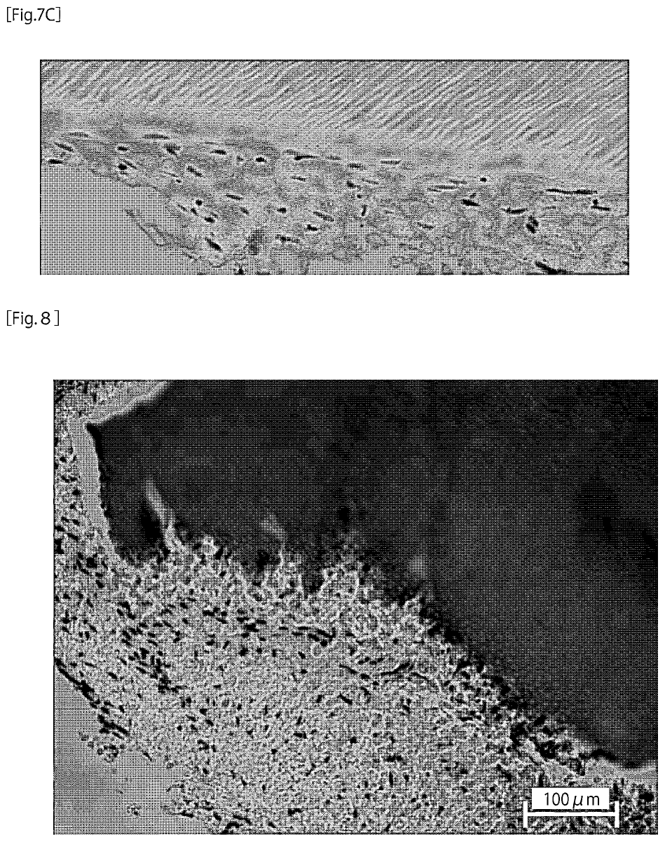

FIGS. 7A to 7C and FIG. 8 are photograms obtained in Example 2 after culturing the pelleted fragmented materials and dental pulp stem cells in which the surfaces of the fragmented materials were processed using (A) 3 wt % EDTA aqueous solution and (B) physiological saline as the control.

As shown in FIGS. 7A to 7C, the adhesion state of dental pulp stem cells to the pellet surface of the fragmented materials processed with the 3 wt % EDTA aqueous solution was better than that of the fragmented materials processed with the control physiological saline solution. Note that the direction of the double-sided arrow illustrated in FIG. 7A indicates the orientation of extension direction of the dentinal tubule, that is, the tubular structure of dentine (hole diameter 1 to 2 μm). FIG. 7C is an enlarged view of region C in FIG. 7B.

From the photographs of FIGS. 7A to 7C, it has been found that the fragmented materials undergo the demineralization process thereby to improve the adhesion properties between the fragmented materials and the dental pulp stem cells.

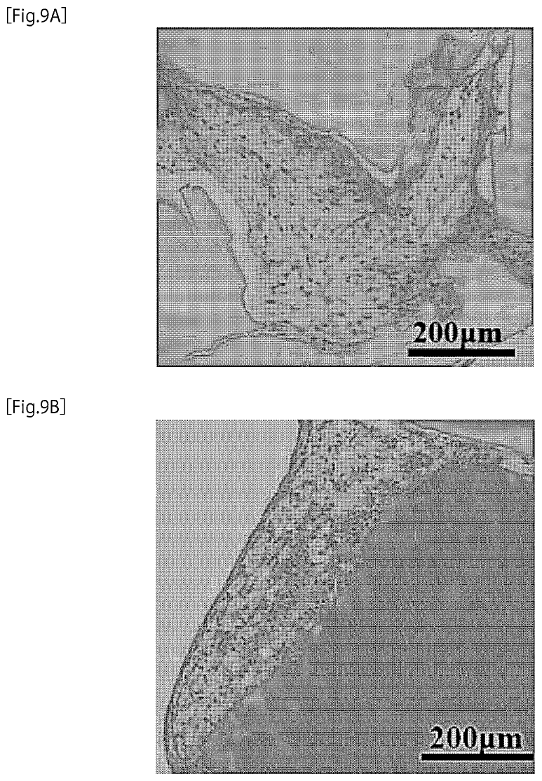

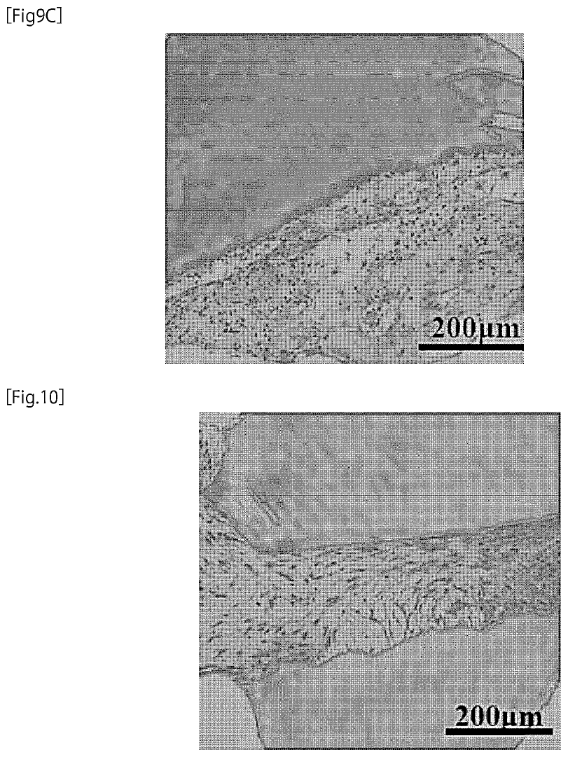

FIGS. 9A, 9B, and 9C are microscope photographs obtained in Example 3 after pelleting and culturing the fragmented materials subjected to the processes (C), (D), and (E) and the dental pulp stem cells. FIG. 10 is a microscope photograph obtained after pelleting and culturing the unprocessed fragmented materials as the control and the dental pulp stem cells.

The dental pulp stem cells are adherent cells, and when they are single cells, they proliferate in a star-like shape. On the other hand, as the dental pulp stem cells become dense, the cell shape elongates to progress the tissue formation. This tissue cell elongation is a characteristic of the dental pulp stem cells. When cultured in a pellet form, the culture environment is dense, and if the surfaces of the fragmented materials are appropriate adhesion scaffolds, the dental pulp stem cells will elongate their shapes, have directionality in a certain direction, and differentiate into tissues.

In the microscope photographs of FIGS. 9A and 9C, it can be confirmed that the cells are elongated to have directionality and the cells are lined up with each other. On the other hand, in the microscope photograph of FIG. 9B, it cannot be clearly confirmed that the cells are elongated to have directionality or the cells are lined up with each other. From these results, it can be said that in order to promote the differentiation of dental pulp stem cells into tubular dentine, bone-like dentine, etc. in a pulp-extirpated tooth, the demineralization process is effective to sufficiently dissolve the enamel of the fragmented materials and expose the dentine on the surfaces of the fragmented materials.

When using the fragmented materials processed with an aqueous hydrochloric acid solution (0.6 normal) for 60 minutes, delamination of the dental pulp stem cells from the pellet surface of the fragmented materials was observed, and the adhesion state was slightly inferior to that of the fragmented materials processed with a 3 wt % EDTA aqueous solution. From these results, it can be said that when using a 0.6 normal hydrochloric acid aqueous solution, the demineralization process is preferably performed for a longer time than 60 minutes, for example, for 10 hours or more, 15 hours or more, or 20 hours or more from the viewpoint of sufficiently dissolving the enamel on the surfaces of the fragmented materials of a tooth.

As shown in FIG. 8, when the physiological saline process using the solution (B) was used as the control in Example 2, no effect of improving the adhesion state of dental pulp stem cells was observed unlike when the fragmented materials were subjected to the demineralization process using the solution (A).

In addition, as shown in FIG. 10, in the undemineralized fragmented materials (F) as the control in Example 3, it has not been recognized that the cells are elongated to have directionality or the cells are lined up with each other.

### INDUSTRIAL APPLICABILITY

The dental composition and dentine culturing method of the present invention can be used for caries treatment, pulp extirpation, and infected root canal treatment.

### DESCRIPTION OF REFERENCE NUMERALS

1: Mixture
2: Resin
3: Fragmented material
4: Composition
5: Dentine substitute
6: Regenerated dentine
7: Regenerated dental pulp
8: Tubular dentine
A to C: Region
r: Root canal diameter
R: Cross-sectional distance

1. A dental composition used for promoting regeneration of dentine, the dental composition comprising a fragmented material of a human tooth.

2. The dental composition according to claim 1, wherein the fragmented material has a particle diameter of 500 to 2000 μm.

3. The dental composition according to claim 1, wherein the fragmented material is subjected to a demineralization process.

4. The dental composition according to claim 1, wherein the fragmented material is subjected to a demineralization process using a 0.4 to 1.0 normal strong acid as a demineralization liquid.

5. The dental composition according to claim 1, wherein the fragmented material is subjected to a demineralization process using an aqueous solution of an ethylenediamine derivative as a demineralization liquid.

6. The dental composition according to claim 1, wherein the fragmented material is immersed in a solution containing an antibacterial agent and then dried.

7. The dental composition according to claim 6, wherein the solution containing the antibacterial agent is an alcohol solution containing silver ions.

8. A dentine culturing method comprising bringing dental pulp cells into contact with a dental composition to induce odontoblasts, the dental composition containing a fragmented material of a human tooth.
